(19) United States
(12) Patent Application Publication
(10) Pub. No.: US 2009/0306483 A1
(43) Pub. Date: Dec. 10, 2009

(54) treatment planning systems

(75) Inventors: Jonas Garding, Stockholm (SE); Per Kjall, Jarna (SE); Par Lidberg, Solna (SE)

Correspondence Address: WESTMAN CHAMPLIN & KELLY, P.A., SUITE 1400, 900 SECOND AVENUE SOUTH, MINNEAPOLIS, MN, 55402, US

(73) Assignee: Elekta AB (publ), Stockholm (SE)

(21) Appl. No.: 12/444,021

(22) PCT Filed: Oct. 2, 2007
(86) PCT No.: PCT/EP2007/008534

§ 371 Date: Apr. 2, 2009

(30) Foreign Application Priority Data
Oct. 3, 2006 (GB) 0619493.0

(51) Int. Cl. A61B 5/00

(52) U.S. Cl. 600/300

(57) ABSTRACT

A treatment planning method comprises the steps of identifying a plurality of treatment locations and an overall dose to be applied to each, for each specific location identifying the global dose applied at that location by doses aimed at different locations, as a proportion of the dose applied at the respective different location, constructing a dose factor matrix containing for each location, the global doses arising from the dose applied at each other location, inverting the dose factor matrix and multiplying it with a prescription matrix containing the overall doses to be applied to each location, thereby to obtain a resultant matrix containing a local dose for each location. This provides a mathematically straightforward method of deriving the necessary doses to be applied to each target so that, including the global doses delivered while irradiating a different target, each target receives the correct total dose. Some locations can be omitted from the treatment planning method, for example if they are located so far from the other locations that they will not have a significant interaction with them. A corresponding treatment planning apparatus is also described.

### FIELD OF THE INVENTION

The present invention relates to treatment planning systems, particularly for use in radiotherapy and in radiosurgery.

### BACKGROUND ART

The Leksell Gamma Knife® is a well-known radiosurgical treatment device, shown illustratively in FIG. 1. It comprises a large number of radioactive Cobalt sources 10 set in a hemispherical array, each arranged to emit a beam of radiation toward the centre 12 of the hemisphere. A generally hemispherical collimator 14 is also provided, provided with an aperture for each source 10, each of which therefore emit a substantially pencil-shaped beam.

At the centre 12, the beams meet and the dose rate is very high. Most other locations within the hemisphere are irradiated by only one source, or possibly no sources, and thus the dose rate is very low. Locations close to the centre 12 will be irradiated by several (although not all of the) sources, as a result of the non-zero thickness of the pencil beams. Thus, the dose distribution consists of a peak at the centre 12, which falls away with increasing distance from the centre. A dose distribution generated by one isocenter is, in the field of radiosurgery, often called a “shot”.

It is becoming increasingly important to provide an effective treatment solution for multiple targets. The basic problem in creating a treatment plan for multiple targets is that the dose contributions of the LGK interact. That is, the dose distribution given to one target will typically contribute some dose to other targets as well.

Complexities can arise when there are multiple targets that require treatment. This has to date been achieved by multiple dose applications so that successive sites are placed at the radiation focus 12 (FIG. 1). As a result, each target site will receive a primary dose, i.e. that delivered when at the device centre 12, together with a background dose, i.e. that delivered incidentally while a different target site is receiving a primary dose. Care must be taken to ensure that the total dose, primary and background, does not exceed the maximum intended dose, particularly outside the target.

The current treatment planning system, Leksell GammaPlan 5.34 (LGP), does allow simultaneous planning of up to 10 different targets. LGP takes the global dose contributions into account and correctly displays the total dose to each target.

### SUMMARY OF THE INVENTION

LGP does not allow a dose to be explicitly prescribed to each individual target. Instead, a global maximum dose is prescribed, which (in practice) means the local prescription to the target in which the global dose maximum occurs. To achieve the intended dose to the other targets, LGP displays (numerically) the maximum dose occurring in each target, and allows the user to change the relative weight (proportional to irradiation time) for the radiation isocenters in each target in 1% or 10% increments. By using this method iteratively, the user may eventually approach the prescribed doses for each target.

This method of balancing dose to multiple targets has several disadvantages; it is manual and time-consuming, and forces the user to keep track of the prescription isodoses outside the treatment planning system—typically on a piece of paper. The final treatment plan does not include details of these prescribed doses, making later audit or verification difficult. The user can only find out by trial and error whether the prescription doses are in fact physically achievable, meaning that if a non-achievable prescription has been requested, the user can waste much time discovering this. The process is also unintuitive and difficult to teach to new users.

Further, the user has no direct and simple way of knowing the relative dose contributions of the local and remote isocenters. This information is important since a strong interaction may mean that the relation between percentage isodoses and dose gradients can differ significantly from the single target case.

The present invention seeks to address these difficulties and move towards a system that overcomes them.

The present invention therefore provides a treatment planning method, comprising the steps of identifying a plurality of treatment locations and an overall dose to be applied to each, for each specific location identifying the global dose applied at that location by doses aimed at different locations, as a proportion of the dose applied at the respective different location, constructing a dose factor matrix containing for each location, the global doses arising from the dose applied at each other location, inverting the dose factor matrix and multiplying it with a prescription matrix containing the overall doses to be applied to each location, thereby to obtain a resultant matrix containing a local dose for each location.

This provides a mathematically straightforward method of deriving the necessary doses to be applied to each target so that, including the global doses delivered while irradiating a different target, each target receives the correct total dose.

The dose factor matrix (i.e. the interaction matrix) is preferably two-dimensional, since there will be a range of target sites, each of which needs to be considered, and each target site will have a contribution offered by its own shots and each other target site. Preferably, the matrix is square, in which case each set of dose factors for each target will include the dose factor applicable to irradiation of the same target; this number will usually be 1, for obvious reasons.

The prescription matrix is preferably one-dimensional, since it needs only contain a single numerical dose for each target.

Some locations can be omitted from the treatment planning method, for example if they are located so far from the other locations that they will not have a significant interaction with them. As the size of the dose factor matrix corresponds to the number of locations in the plurality of treatment locations, a reduction in this number will simplify at least the step of inverting the matrix. This may or may not be useful, depending on the overall size of the matrix and the processing budget available. Thus, we prefer that the plurality of treatment locations is a subset of a larger location set consisting of (i) the plurality of treatment locations and (ii) further locations characterized by weaker interaction, for example because they are spaced from those of the plurality of treatment locations by a distance greater than the spacing of locations within the plurality of treatment locations. Group (ii) therefore consists of locations that are spatially separated from the locations in group (i), and will usually correspond to those locations where there is no significant interaction.

The present invention also relates to a treatment planning apparatus. This can comprise a means for accepting details of a plurality of treatment locations and an overall dose to be applied to each, a processing means arranged to (for each specific location) identify the global dose applied at that location by doses aimed at different locations, as a proportion of the dose applied at the respective different location, construct a dose factor matrix containing for each location, the global doses arising from the dose applied at each other location, invert the dose factor matrix and multiply it with a prescription matrix containing the overall doses to be applied to each location, thereby to obtain a resultant matrix containing a local dose for each location, and an output means arranged to make available the resultant matrix.

The treatment planning apparatus can further comprise a display means. This can be used to display the isodose lines for a planned treatment.

Other preferred aspects of the treatment planning apparatus are as set out above.

### BRIEF DESCRIPTION OF THE DRAWINGS

An embodiment of the present invention will now be described by way of example, with reference to the accompanying figures in which;

FIG. 1 shows in schematic form a two-dimensional section through a Leksell Gamma Knife (“LGK”);

FIG. 2 shows the dose distribution obtained by an LGK; and

FIG. 3 illustrates multiple targets.

### DETAILED DESCRIPTION OF THE EMBODIMENTS

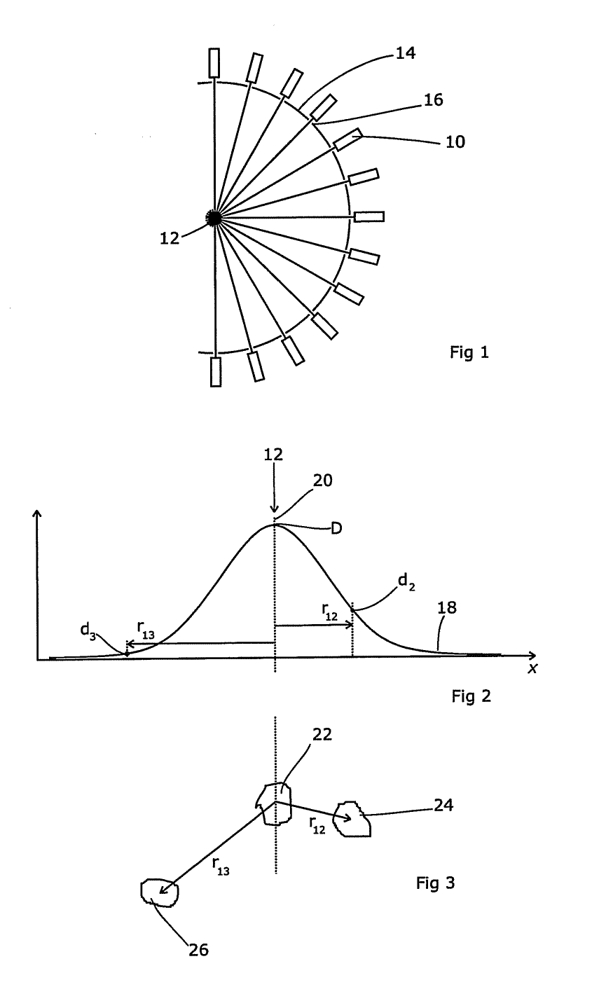

FIG. 1 has been described above and will not be described further.

FIG. 2 shows the dose distribution 18 and how this varies with position (x) in the patient along a line passing through the geometric centre 12 of the LGK. At the centre 12, the dose reaches a peak dose D at 20. This falls off either side of the peak 20, approaching zero at large distances from the centre. FIG. 2 shows a relatively low rate of decline with distance, to illustrate the point.

FIG. 3 shows a case with multiple targets. Thus, it comprises a first target site 22, together with second and third further target sites 24, 26 spaced from the first target site by distances r₁₂ and r₁₃ respectively. Thus, when a primary dose D₁ is being delivered to the first target site 22, the second target site receives a dose d₁₂ and the third target site receives a dose d₁₃.

The distances r₁₂ and r₁₃ and the doses d₁₂ and d₁₃ are shown in FIG. 2. If the distances are not large, which will often be the case with (for example) a metastasized tumour, then d₁₂ and d₁₃ may be non-negligible and will therefore need to be taken into account when planning the primary doses D₂ and D₃ that are to be delivered to the second and third target sites 24, 26. It should also be borne in mind that the delivery of primary dose D₂ (for example) will also involve the delivery of background doses d₂₁ and d₂₃ to the first and third target sites 22, 26 respectively, at distances r₁₂ and r₂₃ respectively. Generally therefore, for a patient with ‘n’ target sites the dose delivered to each site will be made up of one primary dose and (n−1) background doses, i.e. n doses in total, all of which are different and dependent on the relative locations of the sites.

For each site, any adjustment to the primary dose to compensate for the background doses will change the background doses delivered to all other sites, and hence necessitate changing their primary doses, which in turn changes the background dose for which the adjustment was being made in the first place. As n increases therefore, the plan becomes steadily more complex.

According to the invention, the first steps of the workflow for planning the treatment of multiple targets are very similar (or identical) to those involved in planning a single-target treatment. Thus, each target is initially planned in isolation, i.e. as if it were the only target and without taking into account the interaction between targets. For each target, the user selects a local isodose and a prescription dose to that isodose (e.g. 20 Gy to the 50% isodose). An “isodose” is a surface (or a line when viewed in a 2D slice) joining points of equal dose. It allows the effect of the dose distribution to be visually assessed with ease despite the fact that the actual dose varies in three dimensions. Generally, the dose distribution resembles a Gaussian distribution and therefore the rates of change of dose with position is greatest around the 60% isodose line. Clinical users therefore often seek to place an isodose between 40% and 60%, at the physical boundary of the target, to maximise the dose within the target and minimize it outside the target. A dose of 40 Gy to a reference point chosen within a target is therefore often expressed as a dose of (for example) “20 Gy at the 50% isodose”, since the peak dose delivered by such a distribution will be 40 Gy.

The remaining variable is of course the dose amount, reflected in the time for which the radiation is applied and expressed in the treatment planning system as a “weight” applied to each irradiation. The dose is prescribed to each target separately, as in the following example.

TABLE 1
Defining the prescription doses to each target

| Target | Selected target | Prescribed dose | Prescribed dose to |
|---|---|---|---|
| name | isodose | (Gy) to isodose | ref point |
| A | 50% | 20 | 40 |
| B | 40% | 10 | 25 |

To illustrate that the 50% isodose is not mandatory, the isodose chosen for target B is 40% rather than 50%.

The last column is calculated automatically from the chosen isodose and the prescription dose. The reference points have been automatically set to the points of maximum dose from the shots in each target.

The dose prescriptions can be entered before or after the local planning step since (as noted above) they are independent.

The next step is to balance the interaction between the targets. If the separation of the targets is very large so that the interaction is negligible, then the balancing is normally not necessary and the planning is now finished. However, in many cases the shots in one target will contribute significantly to the total dose in other targets, so it is necessary to balance the local and global contribution to achieve the prescribed dose for each target. According to the present invention, this is done automatically as described later. The result may look like this:

TABLE 2
Result of automatic balancing of the local and
remote dose contributions for each target

| | Selected | Prescribed | Prescribed | | Dose from |
|---|---|---|---|---|---|
| Target | target | dose (Gy) to | dose to | Dose from | other |
| name | isodose | isodose | ref point | local shots | targets |
| A | 50% | 20 | 40 | 37.23 | 2.77 |
| B | 40% | 10 | 25 | 13.83 | 11.17 |

In this example, we see that the dose to target A comes mostly from the local shots, so it is likely that the local planning is acceptable without modification.

However, for target B we see that the local and global contributions are of roughly the same magnitude, which is an indication that it will be necessary to review the dose distribution for this target carefully. If the contribution from the other targets (in this case target A) is fairly flat, then there is not much we can do since the dose gradients are then not affected by the local contribution. However, if the global contribution adds a significant gradient, then it may be necessary to compensate by adjusting the shots to target B.

We have now achieved the prescription doses for all targets, but if we find that the isodose lines have moved more than is acceptable, we may need to adjust the plan while considering the total dose. At this point a dose volume histogram (“DVH”) can be computed and evaluated for each target, taking into account the global dose contribution.

In the above discussion, we referred to an automatic process of calculating the global contribution to local doses and adjusting the local doses accordingly in order to achieve the prescribed overall local doses. This will now be described. It is, of course, a non-trivial problem since the modification of the local dose distribution at one target affects the global dose distribution which in turn affects the local dose distribution at every other target; a corresponding adjustment of the local dose distributions at those other targets then affects the dose distribution at that one target, meaning that the adjusted local dose distribution is no longer—in principle—correct.

We define n as the number of targets, and we let each target have a fixed reference point. This is typically the point of maximum dose from the local shots, but need not necessarily be. Let mᵢⱼ be the ratio of the dose to a particular target i that results from the shot(s) delivered to target j. For example, if the reference point for target 3 lies on the 20% isodose line for the local shots to target 5, then m₃₅=0.2.

The total dose tᵢ, to the reference point, to each target can then be expressed as follows:

$$( \begin{matrix}{t}_{1} \\ {t}_{2} \\ {t}_{3} \\ ⋮ \\ {t}_{n}\end{matrix} ) = ( \begin{matrix}{m}_{11} & {m}_{12} & {m}_{13} & … & {m}_{1 n} \\ {m}_{21} & {m}_{22} & {m}_{23} & … & {m}_{2 n} \\ {m}_{31} & {m}_{32} & {m}_{33} & … & {m}_{3 n} \\ ⋮ & ⋮ & ⋮ & ⋱ & ⋮ \\ {m}_{n 1} & {m}_{n 2} & {m}_{n 3} & … & {m}_{nn}\end{matrix} ) ( \begin{matrix}{d}_{1} \\ {d}_{2} \\ {d}_{3} \\ ⋮ \\ {d}_{n}\end{matrix} )$$

where dᵢ, is the maximum dose to target i from the local shots in that target. In matrix format this relation can be expressed more succinctly as

t=Md

By inverting this relation, we can directly and non-iteratively determine the required dose to be given to the local reference point by the shots in each target, to achieve a given prescription dose t:

d=M⁻¹t

This allows the system to display to the user the local contribution dᵢ as well as the global contribution tᵢ−dᵢ, to each target, as shown in Table 2. The advantage of this route is that there are established mathematical algorithms for determining the inverse of a matrix; thus, by choosing to express the problem in this form and by expressing the distance between targets as isodose levels rather than actual distances, we obtain access to a straightforward route to a solution.

In a practical implementation, some variations may be advantageous. For example, each element of d may be scaled by some amount, and the columns of M can be scaled by the inverse of this amount. This variation is mathematically equivalent to the description given above.

If the interaction between targets is strong, and the prescription doses are significantly different, it may happen that the optimum solution contains negative doses d, and therefore cannot be physically realized. This can be illustrated by the following example. The reference points r_{A} and r_{B} in two different targets A and B both lie on the 50% local isodose for the other target. We prescribe 20 Gy to r_{A} and 4 Gy to r_{B}. The mathematical solution is then to give 24 Gy local dose to r_{A}, and −8 Gy local dose to r_{B}:

Total dose to r_{A}=24+0.5*(−8)Gy=20 Gy

Total dose to r_{B}=−8+0.5*24 Gy=4 Gy

Unfortunately, it is not possible to produce negative dose values, so we must settle for a non-optimal solution. This can be defined in a number of ways. One way is to search for the dose that is as close as possible to the prescription dose, while satisfying the constraints that (i) no local dose values are negative, and (ii) the total dose to each target is greater than or equal to the prescription dose. This is a constrained optimization problem, which can be solved e.g. with quadratic programming techniques. For this example, the best we can do is to give 20 Gy to A and 0 Gy to B, resulting in global doses of 20 Gy and 10 Gy to A and B respectively.

Nevertheless, the above-described treatment planning method reveals immediately that there is no realizable solution, by the presence of negative local doses. In an iterative method, it is possible for a clinician to spend a considerable time (with a more complex plan) before giving up; even then there may be a residual doubt as to whether the plan is actually non-realizable or the clinician is simply insufficiently able.

The method described above relies on choosing a reference point for each target such that it does not depend on the dose plan for the other targets. This property allows us to find a closed-form solution (if it exists) to the problem of achieving the prescribed reference dose to each target, as described above.

While planning in local mode, a system operating according to the above description may automatically adjust the 100% reference point to be the point with the maximum contribution from the shot(s) for this target. This means that there may be dose points for which the contribution from other shots means that the effective dose is shown as being above 100%. This is mathematically correct, since the reference point for each local percentage level is the locally applied dose, but it may initially be confusing to some users.

To lessen this potential for confusion, the system can be provided with functionality for finding and displaying the true maximum point within the complete treatment volume and the dose at that point. If users prefer to display isodoses relative to this true maximum, a display mode can be provided that normalizes the percentage isodoses to that global maximum. The selected isodoses should be changed in the same way, so that no isodose lines move; thus only their labels will change (e.g. from 50% to 46%). However, in this mode, the local reference point is still used as the basis for the prescription isodoses. Otherwise, the isodose levels for different targets will be interdependent and any change to one target may necessitate adjustments to other targets in an iterative way, which will be very difficult to fully understand.

To summarize, the system can provide the following three types of isodose display:
1. Local mode: Percentage isodoses for the local shots only. Used to plan the individual doses and to plot the isodose surfaces relative to the target site surfaces.
2. Fine-tuning mode: Percentage isodoses or absolute dose values for dose to a selected target from all shots. Percentage values are based on either the maximum from the local shots or the maximum dose to the target from all shots. Dose curves are only shown within the selected dose matrix (to avoid confusion when panning to a different target without changing the selected target—one could also imagine changing the selected target automatically)
3. Global mode: Absolute dose values for all targets, shown everywhere. This is useful for checking dose to critical structures.

It will of course be understood that many variations may be made to the above-described embodiment without departing from the scope of the present invention.

1. A treatment planning method, comprising the steps of;
identifying a plurality of treatment locations and an overall dose to be applied to each;
for each specific location, identifying the global dose applied at that location by doses aimed at different locations, as a proportion of the dose applied at the respective different location;
applying a mathematical optimization technique to automatically balance the global dose contributions and the local doses applied at each location, in order to obtain the prescribed overall dose to each location.

2. The treatment planning method according to claim 1 in which the mathematical optimization technique comprises;
constructing a dose factor matrix containing for each location, the global dose contributions arising from the local doses applied at each other location;
inverting the dose factor matrix and multiplying it with a prescription matrix containing the overall doses to be applied to each location, thereby to obtain a resultant matrix containing a local dose for each location.

3. The treatment planning method according to claim 2 in which the dose factor matrix is two-dimensional.

4. The treatment planning method according to claim 2 in which the prescription matrix is one-dimensional.

5. The treatment planning method according to claim 1 in which the plurality of treatment locations is a subset of a location set consisting of (i) the plurality of treatment locations and (ii) further locations spaced from those of the plurality of treatment locations by a distance greater than the spacing of locations within the plurality of treatment locations.

6. A treatment Planning Apparatus comprising;
means for accepting details of a plurality of treatment locations and an overall dose to be applied to each;
a processing means arranged to
i. for each specific location, identify the global dose applied at that location by doses aimed at different locations, as a proportion of the dose applied at the respective different location;
ii. construct a dose factor matrix containing for each location, the global doses arising from the dose applied at each other location;
iii. invert the dose factor matrix and multiply it with a prescription matrix containing the overall doses to be applied to each location, thereby to obtain a resultant matrix containing a local dose for each location; and
an output means arranged to make available the resultant matrix.

7. The treatment Planning Apparatus according to claim 6 further comprising a display means.

8. The treatment Planning Apparatus according to claim 7 arranged to display the isodose lines for a planned treatment.

9. The treatment Planning Apparatus according to claim 6 in which the dose factor matrix is two-dimensional.

10. The treatment Planning Apparatus according to claim 6 in which the prescription matrix is one-dimensional.

11. The treatment Planning Apparatus according to claim 6 in which the plurality of treatment locations is a subset of a location set consisting of (i) the plurality of treatment locations and (ii) further locations having a lesser interaction with the locations of the plurality of treatment locations.

12. The treatment Planning Apparatus according to claim 11 in which the further locations are spaced from those of the plurality of treatment locations by a distance greater than the spacing of locations within the plurality of treatment locations.

13. (canceled)

14. (canceled)

15. The treatment planning method according to claim 3 in which the prescription matrix is one-dimensional.

16. The treatment planning method according to claim 2 in which the plurality of treatment locations is a subset of a location set consisting of (i) the plurality of treatment locations and (ii) further locations spaced from those of the plurality of treatment locations by a distance greater than the spacing of locations within the plurality of treatment locations.

17. The treatment planning method according to claim 3 in which the plurality of treatment locations is a subset of a location set consisting of (i) the plurality of treatment locations and (ii) further locations spaced from those of the plurality of treatment locations by a distance greater than the spacing of locations within the plurality of treatment locations.

18. The treatment planning method according to claim 4 in which the plurality of treatment locations is a subset of a location set consisting of (i) the plurality of treatment locations and (ii) further locations spaced from those of the plurality of treatment locations by a distance greater than the spacing of locations within the plurality of treatment locations.

19. The treatment Planning Apparatus according to claim 7 in which the dose factor matrix is two-dimensional.

20. The treatment Planning Apparatus according to claim 8 in which the dose factor matrix is two-dimensional.

21. The treatment Planning Apparatus according to of claim 7 in which the prescription matrix is one-dimensional.

22. The treatment Planning Apparatus according to of claim 8 in which the prescription matrix is one-dimensional.
